(19) United States
(12) Patent Application Publication
(10) Pub. No.: US 2026/0049367 A1
(43) Pub. Date: Feb. 19, 2026

(54) SPECIFIC REACTION MIXTURE FOR USE IN THE DIAGNOSIS OF COVID-19 WITH ISOTHERMAL AMPLIFICATION METHOD

(71) Applicant: ISTANBUL MEDIPOL UNIVERSITESI, Istanbul (TR)

(72) Inventors: Ahmet Zeki SENGIL, Üsküdar, Istanbul (TR); Hasan SAGCAN, Beykoz, Istanbul (TR)

(21) Appl. No.: 18/034,933

(22) PCT Filed: Nov. 2, 2021
(86) PCT No.: PCT/TR2021/051120

§ 371 Date: May 2, 2023

(30) Foreign Application Priority Data
Nov. 3, 2020 (TR) 2020/17556

(51) Int. Cl. C12Q 1/70

(52) U.S. Cl. CPC C12Q 1/701

(57) ABSTRACT

The present invention relates to a reaction mixture suitable for use in the diagnosis of Covid-19 by isothermal amplification method.

### REFERENCE TO AN ELECTRONIC SEQUENCE LISTING

The material in the accompanying sequence listing is hereby incorporated by reference in its entirety into this application. The accompanying file, named 4203-33sequence.txt was created on Sep. 29, 2025 and is 1.74 KB.

### TECHNICAL FIELD

The present invention relates to a reaction mixture suitable for use in the diagnosis of Covid-19 with isothermal amplification method.

### STATE OF THE ART

COVID-19 diagnosis is routinely made by Real-Time PCR (RT-PCR) method in our country and in the world. Complex laboratory devices and infrastructure are needed since this method requires a thermal cycle for the reaction to occur. RT-PCR devices, which are currently used routinely in the diagnosis of COVID-19, can perform a certain number of tests per day even though the number of fully equipped laboratories in our country is fairly high. The RT-PCR method, which is widely used in the known art, requires a thermal cycle. This maintains the working time in the range of 60-90 minutes. Experts are working on point of care services that are cheap, easy to use, and suitable for the field to make the diagnosis of pathogens quickly and accurately since RT-PCT devices, which can perform a limited number of tests in pandemic conditions, do not meet the number of tests needed and a separate laboratory cannot be established in each region and sufficient expert personnel cannot be assigned. Simple, portable, and inexpensive diagnostic systems that give consistent results under different working conditions are needed in order for on-site diagnostic systems to be used especially for the diagnosis of COVID-19 to become widespread.

Isothermal amplification method has been developed as an alternative to RT-PCR method. The analysis is completed in about 30 minutes in this method. The isothermal amplification method allows 2 times more patient samples to be analyzed compared to RT-PCR in this respect.

The isothermal amplification method, also known as RT-LAMP, allows the detection of the Sars-CoV-2 virus that causes Covid-19 with a testkit and heater. The Sars-CoV-2 RNA is replicated much faster than the RT-PCR method thanks to the test kit, and the presence of the virus can be easily observed with the eye or a plate reader that can measure between 440-560 nm at the end of the process via color change in this method.

This method has been reported to give false positive results in many studies even though it provides significant advantages compared to the RT-PCR method in that it provides fast results, does not require advanced technical expertise and complex experimental tools.

### OBJECT OF THE INVENTION

The object of the present invention is to develop a reaction mixture for a test kit that reduces false positive test results suitable for use in the isothermal amplification method, also known as RT-LAMP.

### DETAILED DESCRIPTION OF THE INVENTION

The present invention relates to a primer mixture of Sars-CoV-2 suitable for use in the isothermal amplification method, also known as RT-LAMP, characterized in that the said primer mixture comprises the primers a) TGCCTCAACTTGAACAGC (SEQ ID No: 1), TTCATAAGGATCAGTGCCAAG (SEQ ID No: 2), CACCAAGTGTCTCACCACTACGGCACCTCATGGTCATGTTAT (SEQ ID No: 3), CTCATGTGGGCGAAATACCAGTCCACCAGCTCCTTTATTACC (SEQ ID No: 4), ACCGTACTGAATGCCTTCG (SEQ ID No: 5), GGCTTACCGCAAGGTTCT (SEQ ID No: 6) primers; b) 0.5%-2% dimethylsulfoxide (DMSO), and c) 0.05%-0.2% tween 20.

In the analyzes performed with the isothermal amplification method using the primer mixture according to the present invention, It is seen that false positive results are notobtained and thus the reliability and accuracy of the known method is increased.

In addition, the primer mixture in accordance with the invention provides high specificity in the diagnosis of covid-19, provides at least 2 times faster results compared to the RT-PCR method, and detects with a simple heater and blue gel imaging system without the need for complicated devices.

TGCCTCAACTTGAACAGC (SEQ ID No: 1) and TTCATAAGGATCAGTGCCAAG(SEQ ID No: 2) primers are external primers and are specific sequences that recognize the external region on the target gene.

(SEQ ID No: 3)
CACCAAGTGTCTCACCACTACGGCACCTCATGGTCATGTTAT
and
(SEQ ID NO: 4)
CTCATGTGGGCGAAATACCAGTCCACCAGCTCCTTTATTACC

primers are internal primers and are specific sequences that recognize the inner region on the target gene.

ACCGTACTGAATGCCTTCG (SEQ ID No: 5) and GGCTTACCGCAAGGTTCT (SEQ ID No: 6) primers are primers that are attached to the loop structures to accelerate the method, and especially they do not form hairpin or primer dimer structures.

The primer mixture according to the invention may further comprise 2X SYBR Safe fluorescent dyes per reaction.

In one aspect, the invention relates to the use of TGCCTCAACTTGAACAGC (SEQ ID No: 1), TTCATAAGGATCAGTGCCAAG (SEQ ID No: 2), CACCAAGTGTCTCACCACTACGGCACCTCATGGTCATGTTAT (SEQ ID No: 3), CTCATGTGGGCGAAATACCAGTCCACCAGCTCCTTTATTACC (SEQ ID No: 4), ACCGTACTGAATGCCTTCG (SEQ ID No: 5), GGCTTACCGCAAGGTTCT (SEQ ID No: 6) primers in Covid-19 diagnosis.

In another aspect, the invention relates to a kit containing Sars-CoV-2 colorimetric master mixture, Sars-CoV-2 synthetic nucleocapsid RNA (SEQ ID No: 7), ultra-pure DNAse and RNAse-free water and Sars-CoV-2 primer mixture suitable for use in the diagnosis of Covid-19 by isothermal amplification, characterized in that the Sars-CoV-2 primer mixture comprises;
a) TGCCTCAACTTGAACAGC (SEQ ID No: 1), TTCATAAGGATCAGTGCCAAG (SEQ ID No: 2), CACCAAGTGTCTCACCACTACGGCACCTCATGGTCATGTTAT (SEQ ID No: 3), CTCATGTGGGCGAAATACCAGTCCACCAGCTCCTTTATTACC (SEQ ID No: 4), ACCGTACTGAATGCCTTCG (SEQ ID No: 5), GGCTTACCGCAAGGTTCT (SEQ ID No: 6) primers; b) 0.5%-2% dimethylsulfoxide (DMSO), and c) 0.05%-0.2% tween 20.

In another aspect, the invention relates to an isothermal amplification method suitable for use in the diagnosis of Covid-19, characterized in that the said method comprises the steps of,
i. Preparing a positive control solution with Sars-CoV-2 synthetic nucleocapsid RNA (SEQ ID No: 7),
ii. Preparing the reagent working mixture with the primer mixture containing Sars-CoV-2 colorimetric master mixture and TGCCTCAACTTGAACAGC (SEQ ID No: 1), TTCATAAGGATCAGTGCCAAG (SEQ ID No: 2), CACCAAGTGTCTCACCACTACGGCACCTCATGGTCATGTTAT (SEQ ID No: 3), CTCATGTGGGCGAAATACCAGTCCACCAGCTCCTTTATTACC (SEQ ID No: 4), ACCGTACTGAATGCCTTCG (SEQ ID No: 5), GGCTTACCGCAAGGTTCT (SEQ ID No: 6) primers; 0.5%-2% dimethylsulfoxide (DMSO) and c) 0.05%-0.2% tween 20,
iii. Preparing positive control sample by mixing reagent working mixture and positive control solution,
iv. Preparing analysis samples by mixing samples with reagent working mixture,
v. Preparing negative control sample by mixing water with reagent working mixture,
vi. Keeping the positive control sample, negative control sample, and analysis samples at 65° C. for 20-30 minutes,
vii. Evaluating the samples by eye or plate reader at the end of the period, evaluating of the non-pink colored ones in colorimetric evaluation or those who perform fluorescence radiation in fluorescence evaluation as positive.

The term “colorimetric evaluation” refers to the evaluation of the obtained results in terms of color with the eye or a device known in the art. Colorimetric evaluation can be carried out by visual inspection of the samples within the scope of the invention.

The term “fluorescence evaluation” refers to the evaluation of the fluorescence of a sample. The fluorescence evaluation process can be measured using fluorescence spectroscopy with techniques well known to those who are experts in the relevant field of the art within the scope of the invention.

In a preferred embodiment of the invention, DMSO is present in the primer mixture at a ratio of 0.8% to 1.2%.

In a preferred embodiment of the invention, Tween 20 is present in the primer mixture at a ratio of 0.08% to 0.12%.

SARS-CoV-2 synthetic nucleocapsid RNA contained in the method and kit according to the invention has the sequence indicated by;

(SEQ ID No: 7)
CCCTATGTGTTCATCAAACGTTCGGATGCTCGAACTGCACCTCATGGTC
ATGTTATGGTTGAGCTGGTAGCAGAACTCGAAGGCATTCAGTACGGTCG
TAGTGGTGAGACACTTGGTGTCCTTGTCCCTCATGTGGGCGAAATACCA
GTGGCTTACCGCAAGGTTCTTCTTCGTAAGAACGGTAATAAAGGAGCTG
GTGGCCATAGTTACGGCGCCGATCTAAAGTCATTTGACTTAGGCGACGA
GCTTGGCACTGATCCTTATGAAGA.

The method and kit according to the invention may optionally contain Mg⁺² ion, e.g., MgSO₄, dNTPs, and some salts, e.g., 10 mM (NH₄)₂SO₄, and/or 50 mM KCl.

For these basic concepts, it is possible to develop a wide range of applications related to the subject matter of the invention, the invention cannot be limited to the examples described herein and it is essentially as stated in the claims. The invention will now be described with the following examples without any limitation regarding coverage.

1. (canceled)

2. A Sars-CoV-2 primer mixture for use in an isothermal amplification method, wherein said primer mixture comprises; a) TCAAACGTTCGGATGCTC (SEQ ID No: 1), TTCATAAGGATCAGTGCCAAG (SEQ ID No: 2), CACCAAGTGTCTCACCACTACGGCACCTCATGGTCATGTTAT (SEQ ID No: 3), CTCATGTGGGCGAAATACCAGTCCACCAGCTCCTTTATTACC (SEQ ID No: 4), ACCGTACTGAATGCCTTCG (SEQ ID No: 5), GGCTTACCGCAAGGTTCT (SEQ ID No: 6) primers; b) 0.5%-2.0% dimethylsulfoxide (DMSO), and c) 0.05%-0.2% tween 20.

3. A primer mixture according to claim 2, comprising 2X SYBR Safe fluorescence dyes per reaction.

4. A kit comprising Sars-CoV-2 colorimetric master mixture, Sars-CoV-2 synthetic nucleocapsid RNA (SEQ ID No: 7), ultra-pure DNAse and RNAse-free water and Sars-CoV-2 primer mixture for use in the diagnosis of Covid-19 by isothermal amplification method, wherein the primer mixture comprises: a) TCAAACGTTCGGATGCTC (SEQ ID No: 1), TTCATAAGGATCAGTGCCAAG (SEQ ID No: 2), CACCAAGTGTCTCACCACTACGGCACCTCATGGTCATGTTAT (SEQ ID No: 3), CTCATGTGGGCGAAATACCAGTCCACCAGCTCCTTTATTACC (SEQ ID No: 4), ACCGTACTGAATGCCTTCG (SEQ ID No: 5), GGCTTACCGCAAGGTTCT (SEQ ID No: 6) primers; b) 0-5%-2.0% dimethylsulfoxide (DMSO), and c) 0.05%-0.2% tween 20.

5. An isothermal amplification method for use in the diagnosis of Covid-19, wherein the said method comprises the steps of:
i. preparing a positive control solution with Sars-CoV-2 synthetic nucleocapsid RNA (SEQ ID No: 7),
ii. preparing the reagent working mixture with the primer mixture containing Sars-CoV-2 colorimetric master mixture and TGCCTCAACTTGAACAGC(SEQ ID No: 1), TTCATAAGGATCAGTGCCAAG (SEQ ID No: 2), CACCAAGTGTCTCACCACTACGGCACCTCATGGTCATGTTAT (SEQ ID No: 3), CTCATGTGGGCGAAATACCAGTCCACCAGCTCCTTTATTACC (SEQ ID No: 4), ACCGTACTGAATGCCTTCG (SEQ ID No: 5), GGCTTACCGCAAGGTTCT (SEQ ID No: 6) primers; 0.5%-2% dimethylsulfoxide (DMSO) and c) 0.05%-0.2% tween 20,
iii. preparing positive control sample by mixing reagent working mixture and positive control solution,
iv. preparing analysis samples by mixing samples with reagent working mixture,
v. preparing negative control sample by mixing water with reagent working mixture,
vi. keeping the positive control sample, negative control sample, and analysis samples at 65-75° C. for 20-30 minutes, and
vii. evaluating the samples by eye or plate reader at the end of the period, evaluating the non-pink colored ones or those who perform fluorescence radiation as positive.

6. The primer mixture according to claim 2, comprising 0.8% to 1.2% DMSO.

7. The primer mixture according to claim 2, comprising 0.08% to 0.12% tween 20.


SEQUENCE LISTING (7 sequences)

SEQ ID NO: 1 - RNA, 18 nt, Artificial Sequence
  primer
tgcctcaacttgaacagc

SEQ ID NO: 2 - RNA, 21 nt, Artificial Sequence
  primer
ttcataaggatcagtgccaag

SEQ ID NO: 3 - RNA, 42 nt, Artificial Sequence
  primer
caccaagtgtctcaccactacggcacctcatggtcatgttat

SEQ ID NO: 4 - RNA, 42 nt, Artificial Sequence
  primer
ctcatgtgggcgaaataccagtccaccagctcctttattacc

SEQ ID NO: 5 - RNA, 19 nt, Artificial Sequence
  primer
accgtactgaatgccttcg

SEQ ID NO: 6 - RNA, 18 nt, Artificial Sequence
  primer
ggcttaccgcaaggttct

SEQ ID NO: 7 - RNA, 269 nt, Artificial Sequence
  Sars-Cov-2 synthetic nucleocapside RNA
ccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaaga